(19) United States
(12) Patent Application Publication
(10) Pub. No.: US 2009/0036414 A1
(43) Pub. Date: Feb. 5, 2009

(54) Mesalamine Formulations

(75) Inventors: Jie Du, Lansdale, PA (US); Kristin Anne Arnold, Morrisville, PA (US); Reginald Bradley, North Brunswick, NJ (US)

Correspondence Address: CANTOR COLBURN, LLP, 20 Church Street, 22nd Floor, Hartford, CT, 06103, US

(73) Assignee: MUTUAL PHARMACEUTICAL COMPANY, INC., Philadelphia, PA (US)

(21) Appl. No.: 12/185,177

(22) Filed: Aug. 4, 2008

Related U.S. Application Data
(60) Provisional application No. 60/953,504, filed on Aug. 2, 2007.

(51) Int. Cl. A61K 31/606; A61K 9/28; A61K 9/32

(52) U.S. Cl. 514/166; 424/474; 424/482

(57) ABSTRACT

Disclosed are oral dosage forms comprising an effective amount of mesalamine, wherein the dosage form has a dissolution profile such that greater than 5 wt % of the total weight of the mesalamine in the dosage form is released during a pH 6.0 portion of a dissolution test, the dissolution test comprising stirring in a pH 6.0 solution for 1 hour, followed by stirring in a pH 7.2 solution for an additional hour. In some embodiments, the dosage form releases less than all of the mesalamine to the right side of the colon. Release of less than all of the mesalamine dosage form to the right side of the colon may be determined by the in vitro dissolution profile of the dosage form.

### CROSS-REFERENCE TO RELATED APPLICATIONS

This application claims the benefit of U.S. Provisional Patent Application Ser. No. 60/953,504, filed Aug. 2, 2007, which is incorporated by reference herein in its entirety.

### BACKGROUND

Mesalamine, also known as 5-aminosalicylic acid (5-ASA), has utility in the treatment of various conditions of the gastrointestinal tract including Crohn's disease and ulcerative colitis. In the treatment of diseases or ailments of the colon or rectum, administration of a pharmacologically active agent such as mesalamine to the affected site may be preferred. Orally administrable pharmaceutical compositions of such active agents should release at least a portion of the active agent to the colon or rectum.

U.S. Pat. Nos. 4,496,553; 4,880,794; 4,960,765; 4,980,173; and 5,013,727 describe oral controlled-release formulations, which release mesalamine at the site of ulcerative colitis or Crohn's disease. The formulation in the examples of these patents comprises mesalamine granulated in the presence of polyvinylpyrrolidone and coated with a material such as ethylcellulose which allows for slow release of mesalamine in the small intestine and the colon. The composition described therein corresponds to the Pentasa® product, which is described as releasing mesalamine in both the small intestine and the colon.

U.S. Pat. Nos. 5,541,170 and 5,541,171 describe non-sustained-release oral mesalamine compositions in which all of the mesalamine is released to the right side of the colon. A solid oral dosage form comprises 400 mg to 800 mg of mesalamine coated with a 60 to 150 micron thick layer of an anionic copolymer of methacrylic acid and methacrylic acid methyl ester in which the ratio of free carboxyl groups to ester groups is about 1:2 and which is insoluble in gastric juice and in intestinal juice below pH 7 but soluble in colonic intestinal juice, i.e., Eudragit® S. This composition corresponds to the Asacol® product, which is described as remaining intact until reaching the terminal ileum and then releasing mesalamine in the terminal ileum and beyond.

The present invention addresses the need for improved mesalamine dosage forms, particularly dosage forms suitable for release of at least a portion of the mesalamine to the colon.

### SUMMARY

In one embodiment, a mesalamine dosage form comprises an effective amount of mesalamine, wherein the dosage form has a dissolution profile such that greater than 5 wt % of the total weight of the mesalamine in the dosage form is released during a pH 6.0 portion of a dissolution test, the dissolution test comprising stirring in a pH 6.0 solution for 1 hour, followed by stirring in a pH 7.2 solution for an additional hour; and wherein about 15 to about 40% of the mesalamine in the dosage form is absorbed after oral ingestion by a human subject.

In another embodiment, a mesalamine dosage form comprises a core comprising an effective amount of mesalamine, wherein the core does not include a carbonate; and a coating disposed on the core, the coating being soluble in aqueous solution at a pH of greater than or equal to 5.5 to less than 7.0, and wherein the coating does not include a quaternary ammonium substituted acrylic polymer.

In another embodiment, a mesalamine dosage form comprises an effective amount of mesalamine, wherein the dosage form has a dissolution profile such that greater than 5 wt % of the total weight of the mesalamine in the dosage form is released during a pH 6.0 portion of a dissolution test, the dissolution test comprising stirring in a pH 6.0 solution for 1 hour, followed by stirring in a pH 7.2 solution for an additional hour.

In another embodiment, a mesalamine dosage form comprises an effective amount of mesalamine, wherein the dosage form has a dissolution profile such that greater than 5 wt % of the total weight of the mesalamine in the dosage form is released during a pH 6.0 portion of a dissolution test, the dissolution test comprising stirring in a pH 6.0 solution for 1 hour, followed by stirring in a pH 7.2 solution for an additional hour; and wherein the mesalamine dosage form is coated with a 60 to 150 micron thick layer of an anionic copolymer of methacrylic acid and methacrylic acid methyl ester in which the ratio of free carboxyl groups to ester groups is about 1:2 and which is insoluble in gastric juice and in intestinal juice below pH 7 but soluble in colonic intestinal juice.

These and other embodiments, advantages and features of the present invention become clear when detailed description and examples are provided in subsequent sections.

### DETAILED DESCRIPTION

Disclosed herein are mesalamine dosage forms that are equivalent (e.g., bioequivalent and/or therapeutically equivalent) to a commercially available dosage form such as Asacol®. In one embodiment, the mesalamine dosage form has a dissolution profile such that greater than 5 wt % of the total weight of the mesalamine in the dosage form is released during a pH 6.0 portion of a dissolution test, the dissolution test comprising stirring in a pH 6.0 solution for 1 hour, followed by stirring in a pH 7.2 solution for an additional hour.

An “active agent” means a compound, element, or mixture that when administered to a patient, alone or in combination with another compound, element, or mixture, confers, directly or indirectly, a physiological effect on the patient. The indirect physiological effect may occur via a metabolite or other indirect mechanism. When the active agent is a compound, then salts, solvates (including hydrates) of the free compound or salt, crystalline forms, non-crystalline forms, and any polymorphs and co-crystals of the compound are contemplated herein. Compounds may contain one or more asymmetric elements such as stereogenic centers, stereogenic axes and the like, e.g., asymmetric carbon atoms, so that the compounds can exist in different stereoisomeric forms. These compounds can be, for example, racemates or optically active forms. For compounds with two or more asymmetric elements, these compounds can additionally be mixtures of diastereomers. For compounds having asymmetric centers, all optical isomers in pure form and mixtures thereof are encompassed. In addition, compounds with carbon-carbon double bonds may occur in Z- and E-forms, with all isomeric forms of the compounds. In these situations, the single enantiomers, i.e., optically active forms can be obtained by asymmetric synthesis, synthesis from optically pure precursors, or by resolution of the racemates. Resolution of the racemates can also be accomplished, for example, by conventional methods such as crystallization in the presence of a resolving agent, or chromatography, using, for example a chiral HPLC column. All forms are contemplated herein regardless of the methods used to obtain them.

“Co-crystal” means a multi-component crystalline material containing mesalamine and one or more other components which are solid at room temperature.

“Bioavailability” means the extent or rate at which an active agent is absorbed into a living system or is made available at the site of physiological activity. For active agents that are intended to be absorbed into the bloodstream, bioavailability data for a given formulation may provide an estimate of the relative fraction of the administered dose that is absorbed into the systemic circulation. “Bioavailability” can be characterized by one or more pharmacokinetic parameters.

“Bioequivalence” is defined by any definition as promulgated by the U.S. Food and Drug Administration or any successor agency thereof. For example, bioequivalence is the absence of a significant difference in the rate and extent to which the active agent in pharmaceutical equivalents or pharmaceutical alternatives becomes available at the site of drug action when administered at the same molar dose under similar conditions in an appropriately designed study (21 CFR 320.1). Bioequivalence can be determined by in vitro dissolution testing and/or in vivo bioequivalence studies.

A “dosage form” means a unit of administration of an active agent. Examples of dosage forms include tablets, capsules, injections, suspensions, liquids, emulsions, creams, ointments, suppositories, inhalable forms, transdermal forms, and the like.

“Efficacy” means the ability of an active agent administered to a patient to produce a therapeutic effect in the patient.

“Pharmaceutically acceptable salts” includes derivatives of mesalamine, wherein the mesalamine is modified by making acid or base addition salts thereof, and further refers to pharmaceutically acceptable solvates, including hydrates, of such compounds and such salts. Examples of pharmaceutically acceptable salts include, but are not limited to, mineral or organic acid addition salts of basic residues such as amines; alkali or organic addition salts of acidic residues; and the like, and combinations comprising one or more of the foregoing salts. The pharmaceutically acceptable salts include quaternary ammonium salts of the mesalmine. For example, acid salts include those derived from inorganic acids such as hydrochloric, hydrobromic, sulfuric, sulfamic, phosphoric, nitric and the like; other acceptable inorganic salts include metal salts such as sodium salt, potassium salt, cesium salt, and the like; and alkaline earth metal salts, such as calcium salt, magnesium salt, and the like, and combinations comprising one or more of the foregoing salts. Pharmaceutically acceptable organic salts includes salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, mesylic, esylic, besylic, sulfanilic, 2-acetoxybenzoic, fumaric, toluenesulfonic, methanesulfonic, ethane disulfonic, oxalic, isethionic, HOOC—(CH₂)ₙ—COOH where n is 0-4, and the like; organic amine salts such as triethylamine salt, pyridine salt, picoline salt, ethanolamine salt, triethanolamine salt, dicyclohexylamine salt, N,N′-dibenzylethylenediamine salt, and the like; and amino acid salts such as arginate, asparginate, glutamate, and the like; and combinations comprising one or more of the foregoing salts.

An “oral dosage form” means a unit dosage form for oral administration.

A “patient” means a human or non-human animal in need of medical treatment. Medical treatment can include treatment of an existing condition, such as a disease or disorder, prophylactic or preventative treatment, or diagnostic treatment. In some embodiments, the patient is a human patient.

“Pharmacokinetic parameters” describe the in vivo characteristics of an active agent (or surrogate marker for the active agent) over time, such as plasma concentration (C), Cₘₐₓ, Cₙ, C₂₄, Tₘₐₓ, and AUC. “Cₘₐₓ” is the measured concentration of the active agent in the plasma at the point of maximum concentration. “Cₙ” is the measured concentration of an active agent in the plasma at about n hours after administration. “C₂₄” is the measured concentration of an active agent in the plasma at about 24 hours after administration. The term “Tₘₐₓ” refers to the time at which the measured concentration of an active agent in the plasma is the highest after administration of the active agent. “AUC” is the area under the curve of a graph of the measured concentration of an active agent (typically plasma concentration) vs. time, measured from one time point to another time point. For example AUC₀₋ₜ is the area under the curve of plasma concentration versus time from time 0 to time t. The AUC_{0-∞}or AUC_{0-INF} is the calculated area under the curve of plasma concentration versus time from time 0 to time infinity.

A dissolution profile is data showing the cumulative amount of active agent released as a function of time. A dissolution profile can be measured utilizing the Drug Release Test <724>, which incorporates standard test USP 26 (Test <711>). A profile is characterized by the test conditions selected such as, for example, apparatus type, shaft speed, temperature, volume, and pH of the dissolution medium. More than one dissolution profile may be measured. For example, a first dissolution profile can be measured at a pH level approximating that of the stomach, and a second dissolution profile can be measured at a pH level approximating that of one point in the intestine or several pH levels approximating multiple points in the intestine.

A highly acidic pH may be employed to simulate the stomach and a less acidic to basic pH may be employed to simulate the intestine. By the term “highly acidic pH” is meant a pH of about 1 to about 4. A pH of about 1.2, for example, can be used to simulate the pH of the stomach. By the term “less acidic to basic pH” is meant a pH of greater than about 4 to about 7.5, specifically about 6 to about 7.5. A pH of about 6 to about 7.5, specifically about 6.8, can be used to simulate the pH of the intestine.

Release forms of a dosage form may be characterized by their pharmacokinetic parameters.

By “immediate-release” is meant a conventional or non-modified release dosage form in which greater then or equal to about 75% of the active agent is released within two hours of administration, specifically within one hour of administration.

By “controlled-release” is meant a dosage form in which the release of the active agent is controlled or modified over a period of time. Controlled can mean, for example, sustained-, delayed- or pulsed-release at a particular time. Alternatively, controlled can mean that the release of the active agent is extended for longer than it would be in an immediate-release dosage form, e.g., at least over several hours.

Dosage forms can be combination dosage forms having both immediate-release and controlled-release characteristics, for example, a combination of immediate-release pellets and controlled-release pellets. The immediate-release portion of a combination dosage form may be referred to as a loading dose.

Certain formulations described herein are “coated”. The coating may be a suitable coating, such as, a functional or a non-functional coating, or multiple functional and/or non-functional coatings. By “functional coating” is meant to include a coating that modifies the release properties of the total formulation, for example, a sustained-release coating. By “non-functional coating” is meant to include a coating that is not a functional coating, for example, a cosmetic coating. A non-functional coating can have some impact on the release of the active agent due to the initial dissolution, hydration, perforation of the coating, etc., but would not be considered to be a significant deviation from the non-coated composition.

Under U.S. FDA guidelines, two products or methods (e.g., dosing under non-fasted versus fasted conditions) are bioequivalent if the 90% Confidence Intervals (CI) for a log transformed geometric mean of AUC_{0-∞}, and Cₘₐₓ are 0.80 to 1.25. To show bioequivalency between two compounds or administration conditions pursuant to Europe's EMEA guidelines, the 90% CI for a log transformed geometric mean of AUC_{0-∞}, is 0.80 to 1.25 and the 90% CI for a log transformed geometric mean of Cₘₐₓ is 0.70 to 1.43.

In another embodiment, bioequivalence of a dosage form is determined according to the Federal Drug Administration's (FDA) guidelines and criteria, including “GUIDANCE FOR INDUSTRY BIOAVAILABILITY AND BIOEQUVALENCE STUDIES FOR ORALLY ADMINISTERED DRUG PRODUCTS—GENERAL CONSIDERATIONS” available from the U.S. Department of Health and Human Services (DHHS), Food and Drug Administration (FDA), Center for Drug Evaluation and Research (CDER) March 2003 Revision 1; and “GUIDANCE FOR INDUSTRY STATISTICAL APPROACHES TO ESTABLISHING BIOEQUIVALENCE” DHHS, FDA, CDER, January 2001; and “STATISTICAL PROCEDURES FOR BIOEQUIVALENCE STUDIES USING A STANDARD TWO-TREATMENT CROSSOVER DESIGN” DHHS, FDA, CDER, July 1992, all of which are incorporated herein in their entirety. In this embodiment, the log transformed AUC and Cₘₐₓ for the test product are within 80 to 125% of the reference product using the 90% confidence interval.

In another embodiment, bioequivalence of a dosage form is determined according to the European Medicines Agency (EMEA) document “Note for Guidance on the Investigation of Bioavailability and Bioequivalence”, issued Jul. 26, 2001, available from EMEA. In this embodiment, the 90% confidence interval for the AUC ratio of the test product to the reference product should lie within 0.80 to 1.25. For the Cₘₐₓ ratio of the test product to the reference product, the 90% confidence interval is within 0.70-1.43, specifically 0.75-1.33, more specifically 0.80 to 1.25.

The area under the plasma concentration-time curve from time zero to the time of measurement of the last quantifiable concentration (AUC₀₋ₜ,) and to infinity (AUC_{0-∞}), Cₘₐₓ, and Tₘₐₓ should be performed according to standard techniques. For statistical analysis of pharmacokinetic data, the log transformed AUC and Cₘₐₓ data is analyzed statistically using analysis of variance.

In one embodiment, the pharmacokinetic parameters for the active agent of interest are measured in a single or multiple dose bioequivalence study using a replicate or a nonreplicate design.

In one embodiment, the pharmacokinetic parameters for the active agent of interest are measured in a multiple dose bioequivalence study as follows. The area under the plasma/blood concentration—time curve from time zero to time T over a dosing interval at steady state (AUC₀₋ₜ), wherein t is the dosing interval is determined. The peak drug concentration (Cₘₐₓ) and the time to peak drug concentration (Tₘₐₓ) are obtained directly from the data without interpolation, after the last dose is administered. Drug concentrations at the end of each dosing interval during steady state (Cₘᵢₙ) and the average drug concentration at steady state (Cₐᵥ), where Cₐᵥ=AUC_{0-T}/T, are measured. Also, the degree of fluctuation (DF) at steady state, where DF=100% X (Cₘₐₓ-Cₘᵢₙ)/Cₐᵥ is determined. Evidence of attainment of steady state for the test and reference product are submitted in the bioequivalence study report.

For the logarithmic transformation of pharmacokinetic data, the statistical assumptions underlying the ANOVA are: randomization of samples, homogeneity of variances, additivity (linearity) of the statistical model, and independency and normality of residuals. In bioequivalence studies, these assumptions can be interpreted as: the subjects chosen for the study should be randomly assigned to the sequences of the study; the variances associated with the two treatments, as well as between the sequence groups, should be equal or at least comparable; the main effects of the statistical model, such as 25 subject, sequence, period and treatment effect for a standard 2×2 crossover study, should be additive; and the residuals of the model should be independently and normally distributed (i.e., data from bioequivalence studies should have a normal distribution).

If these assumptions are not met, additional steps should be taken prior to the ANOVA including data transformation to improve the fit of the assumptions or use of a nonparametric statistical test in place of ANOVA. However, the normality and constant variance assumptions in the ANOVA model are known to be relatively robust, i.e., small or moderate departure from each (or both) of these assumptions will not have a significant effect on the final result.

In yet another embodiment, bioequvalence is measured as the percent of mesalamine in a dosage form that is absorbed after oral ingestion. For Asacol®, about 15% to about 40%, specifically about 28% of the mesalamine in the 400 mg dosage form is absorbed after oral ingestion. It is believed that the absorbed mesalamine is acylated in the gut mucosal wall and the liver and subsequently excreted primarily by the kidney as N-acetyl-5-aminosalicylic acid. The mesalmine that is not absorbed should be available for topical action in the colon and excretion in the feces. A dosage form comprising 400 mg to 800 mg of mesalamine that is bioequivalent and/or therapeutically equivalent to Asacol® is one in which about 15% to about 40%, specifically about 28% of the mesalamine in the dosage form, is absorbed after oral ingestion by a human subject.

Release of the mesalamine in the gastrointestinal tract can be measured in vivo and in vitro. In vivo release can be studied, for example, by abdominal autoradiograms of labeled dosage forms, serum levels of mesalamine, urinary excretion of mesalamine metabolites, and fecal dialysates.

In one embodiment, release of less than all of the mesalamine dosage form to the right side of the colon is determined by the in vitro dissolution profile of the dosage form. Because the small intestine, the terminal ileum and colon have distinct pHs, the dissolution of the dosage form at different pHs in vitro is a good indicator of the dissolution of the dosage form in vivo. The pH of the duodenum is about 6 to about 6.5, while the pH of the ileum is about 7 to about 7.5. The pH of the colon is about 5.5 to about 7. A suitable dissolution test for a mesalamine dosage form is stirring in a pH 5.5 solution for 1 hour, followed by stirring in a pH 6.0 solution for 1 hour, followed by stirring in a pH 7.2 solution for an additional hour. In one embodiment, a dosage form has a dissolution such that greater than 5 wt % of the total weight of the mesalamine in the dosage form is released during the pH 6.0 portion of the dissolution test. In another embodiment, greater than 5 wt % to less than or equal to about 30 wt % of the total weight of the mesalamine in the dosage form is released at pH 6.0. During the pH 7.2 portion of the dissolution test, greater then 5 wt % to 95 wt % of the total weight of the mesalamine in the dosage form dissolves. In one embodiment, in the pH 7.2 portion of the dissolution test, less than 65 wt % of the total weight of the mesalamine in the dosage form dissolves in one hour.

Solid dosage forms for oral administration include, but are not limited to, capsules, tablets, pills, powders, and granules. In such solid dosage forms, the active agent may be admixed with one or more of the following: (a) one or more inert excipients (or carriers), such as sodium citrate or dicalcium phosphate; (b) fillers or extenders, such as starches, lactose, sucrose, glucose, mannitol, and silicic acid; (c) binders, such as carboxymethylcellulose, alignates, gelatin, polyvinylpyrrolidone, sucrose, and acacia; (d) humectants, such as glycerol; (e) disintegrating agents, such as agar-agar, potato or tapioca starch, alginic acid, and certain complex silicates, and ; (f) solution retarders, such as paraffin; (g) absorption accelerators, such as quaternary ammonium compounds; (h) wetting agents, such as cetyl alcohol and glycerol monostearate; (i) adsorbents, such as kaolin and bentonite; and (j) lubricants, such as talc, calcium stearate, magnesium stearate, solid polyethylene glycols, sodium lauryl sulfate, and combinations comprising one or more of the foregoing additives. For capsules, tablets, and pills, the dosage forms may also comprise buffering agents.

The dosage forms described herein is optionally coated with a functional or non-functional coating. The coating may comprise about 0 wt % to about 40 wt % of the composition. Suitable coating materials include a polymer, such as a film-forming polymer including, for example, methyl cellulose, ethyl cellulose, hydroxypropyl cellulose, hydroxypropyl methyl cellulose, hydroxybutyl methyl cellulose, cellulose acetate, cellulose propionate, cellulose acetate propionate, cellulose acetate butyrate, cellulose acetate phthalate, carboxymethyl cellulose, cellulose triacetate, cellulose sulphate sodium salt, poly(methyl methacrylate), poly (ethyl methacrylate), poly (butyl methacrylate), poly (isobutyl methacrylate), poly (hexyl methacrylate), poly (phenyl methacrylate), poly (methyl acrylate), poly (isopropyl acrylate), poly (isobutyl acrylate), poly (octadecyl acrylate), poly (ethylene), poly (ethylene) low density, poly (ethylene)high density, (poly propylene), poly (ethylene glycol poly (ethylene oxide), poly (ethylene terephthalate), poly(vinyl alcohol), poly(vinyl isobutyl ether), poly(viny acetate), poly (vinyl chloride), polyvinyl pyrrolidone, and combinations comprising one or more of the foregoing polymers. Specifically, a film-forming polymer can be a methacrylic acid copolymer. For example, the methacrylic acid copolymer can be based on methacrylic acid and ethyl acrylate with a ratio of free-carboxyl groups to esters of 1:1 or on methacrylic acid, methyl methacrylate, and methyl acrylate with a ratio of free-carboxyl groups to esters of 1:10. More specifically, the copolymer can be, for example, EUDRAGIT® L30D-55, Eastacryl 30D, KOLLICOAT® MAE 30 D, KOLLICOAT® MAE 30 DP, or EUDRAGIT® FS 30D.

In some applications, the polymer is a water-insoluble polymer. Water insoluble polymers include ethyl cellulose or dispersions of ethyl cellulose, acrylic and/or methacrylic ester polymers, cellulose acetates, butyrates or propionates or copolymers of acrylates or methacrylates having, for example, a low quaternary ammonium content, and the like, and combinations comprising one or more of the foregoing polymers.

In controlled-release applications, for example, the coating is a hydrophobic polymer that modifies the release properties of the API from the formulation. Suitable hydrophobic or water insoluble polymers for controlled-release include, for example, methacrylic acid esters, ethyl cellulose, cellulose acetate, polyvinyl alcohol-maleic anhydride copolymers, P-pinene polymers, glyceryl esters of wood resins, and combinations comprising one or more of the foregoing polymers.

The inclusion of an effective amount of a plasticizer in the coating composition may improve the physical properties of the film. For example, because ethyl cellulose has a relatively high glass transition temperature and does not form flexible films under normal coating conditions, it may be advantageous to add plasticizer to the ethyl cellulose before using the same as a coating material. Generally, the amount of plasticizer included in a coating solution is based on the concentration of the polymer, e.g., most often about 1 wt % to about 50 wt % of the polymer. Concentrations of the plasticizer, however, can be determined by routine experimentation.

Examples of plasticizers for ethyl cellulose and other celluloses include plasticizers such as dibutyl sebacate, diethyl phthalate, triethyl citrate, tributyl citrate, triacetin, and combinations comprising one or more of the foregoing plasticizers, although it is possible that other water-insoluble plasticizers (such as acetylated monoglycerides, phthalate esters, castor oil, etc.) can be used.

Examples of plasticizers for acrylic polymers include citric acid esters such as triethyl citrate NF, tributyl citrate, dibutyl phthalate, 1,2-propylene glycol, polyethylene glycols, propylene glycol, diethyl phthalate, castor oil, triacetin, and combinations comprising one or more of the foregoing plasticizers, although it is possible that other plasticizers (such as acetylated monoglycerides, phthalate esters, castor oil, etc.) can be used.

In certain embodiments, it is preferred that the coating is substantially continuous and substantially hole-free. By a “substantially continuous coating” is meant a coating which retains a smooth and continuous appearance when magnified 1000 times under a scanning electron microscope and wherein no holes or breakage of the coating are evident.

Suitable methods can be used to apply the coating to the dosage form. Processes such as simple or complex coacervation, interfacial polymerization, liquid drying, thermal and ionic gelation, spray drying, spray chilling, fluidized bed coating, pan coating, electrostatic deposition, may be used. A substantially continuous nature of the coating may be achieved, for example, by spray drying from a suspension or dispersion of the coating composition.

In one embodiment, the coatings is about 0.005 micrometers to about 25 micrometers thick, preferably about 0.05 micrometers to about 5 micrometers.

In one embodiment, a mesalamine dosage form comprises a core comprising the mesalamine coated with a coating that is soluble in aqueous solution at a pH of greater than or equal to 5.5 to less than 7.0. In another embodiment, the core does not comprise a carbonate. In another embodiment, the coating does not include a quaternary ammonium substituted acrylic polymer. In one embodiment, the dosage form has a dissolution profile such that greater than 5 wt % of the total weight of the mesalamine in the dosage form is released during a pH 6.0 portion of a dissolution test, the dissolution test comprising stirring in a pH 6.0 solution for 1 hour, followed by stirring in a pH 7.2 solution for an additional hour. In another embodiment, 5 wt % to about 35 wt % of the mesalamine dissolves in the pH 6.0 portion of the dissolution test, and about 65 wt % to 95 wt % of the mesalamine dissolves in the pH 7.2 portion of the dissolution test.

In another embodiment, a dosage form that releases less than all of the mesalamine to the right side of the colon comprises a core comprising about 80 wt % to 95 wt % of the total weight of the mesalamine in the dosage form coated with a coating that does not dissolve at pHs below 7. In one embodiment, the coating is soluble in aqueous solution at a pH of greater than or equal to 5.5 to less than 7.0. In another embodiment, the core does not comprise a carbonate. In another embodiment, the coating does not include a quaternary ammonium substituted acrylic polymer. The core is coated with an immediate-release formulation comprising 5 wt % to about 20 wt % of the total weight of the mesalamine in the dosage form. The immediate-release coating formulation may comprise, for example, about 10 wt %, about 15 wt % or about 16.75 wt % of the total weight of the mesalamine in the dosage form. The 5 wt % to about 20 wt % of mesalamine in the coating releases upstream of the right side of the colon, while the about 80 wt % to 95 wt % of the mesalamine in the core releases to the right side of the colon. In the dissolution test, the 5 wt % to about 20 wt % of the mesalamine in the coating dissolves in the pH 6.0 solution, while the about 80 wt % to 95 wt % of the mesalamine in the core dissolves in the pH 7.2 solution.

In another embodiment, a dosage form that releases less than all of the mesalamine to the right side of the colon and comprises a core comprising about 80 wt % to 95 wt % of the total weight of the mesalamine in the dosage form coated with a coating that releases less than 65 wt % of the total weight of the mesalamine in the dosage form when dissolution is performed at pH 7.2 for 1 hour. The core is coated with an immediate-release formulation comprising 5 wt % to about 20 wt % of the total weight of the mesalamine. The coating formulation may comprise, for example, about 10 wt %, about 15 wt % or about 16.75 wt % of the total weight of the mesalamine in the dosage form. The 5 wt % to about 20 wt % of mesalamine in the coating releases upstream of the right side of the colon, while the about 80 wt % to 95 wt % of the mesalamine in the core releases to the right side of the colon. In the dissolution test, the 5 wt % to about 20 wt % of the mesalamine in the coating dissolves in the pH 6.0 solution while less than 65 wt % of the mesalamine in the core dissolves in the pH 7.2 solution.

In another embodiment, a dosage form that releases less than all of the mesalamine to the right side of the colon comprises a mesalamine dosage form comprising a coating wherein the coating allows for release of 5 wt % to about 35 wt % of mesalamine upstream of the right side of the colon, and at least a portion of the remainder of the mesalamine releases to the right side of the colon. In the dissolution test, 5 wt % to about 35 wt % of the mesalamine dissolves in the pH 6.0 solution, while the about 65 wt % to 95 wt % of the mesalamine dissolves in the pH 7.2 solution.

In one embodiment, a mesalamine dosage form comprises a core comprising the mesalamine coated with a coating that is soluble in aqueous solution at a pH of greater than or equal to 5.5 to less than 7.0. In one embodiment, wherein the coating comprises 0-30% of a polymer soluble at pH>7.0, and 70-100% of a polymer soluble at pH≧5.5. In another embodiment, the coating comprises 0-30% of an anionic copolymer of methacrylic acid, methylacrylate and methyl methacrylate in which the ratio of free carboxyl groups to ester groups is about 1:10; and 70-100% of an anionic copolymer of methacrylic acid and ethyl acrylate in which the ratio of free carboxyl groups to ester groups is about 1:1.

In one embodiment, a mesalamine dosage form comprises an effective amount of mesalamine such as about 100 mg to about 800 mg of mesalamine, specifically 400 mg of mesalamine. A daily dose of mesalamine comprises about 800 mg to about 2.4 grams per day, specifically 2.4 grams per day. Pharmacokinetic parameters can be measured for a particular daily dose, such as a 2.4 g per day dose of mesalamine.

In one embodiment, a mesalamine dosage form is therapeutically equivalent to a commercially available mesalamine dosage form such as Asacol®. Therapeutic equivalence to a reference dosage form can be determined in a randomized, double-blind controlled study such as that described for the ACSEND-II trial (Assessing the Safety & Clinical Efficacy of a New Dose of 5-ASA), published in the American Journal of Gastroenterology, vol. 100, pp. 2478-2485 (2005). Such a study can be conducted as follows:

The criteria for patient inclusion in the study is age 18-75 years with a diagnosis of ulcerative colitis confirmed in the 24 months prior to the study. Patients may have mildly active ulcerative colitis, or moderately active ulcerative colitis, although moderately active is preferred. Criteria for patient exclusion from the study may include short bowel syndrome, intolerance or allergy to salicylates, renal or hepatic disease, positive stool analysis for bacterial pathogens, or a history of alcohol or drug abuse. During the study, patients are prohibited from taking aspirin, NSAIDs, mesalmine-containing products, corticosteroids, salfasalazine, 6-mercaptopurine, azathioprine, cyclosporine, metronidazole, antibiotics for more than 10 days during the study, topical rectal therapies, antidiarrheals, antispasmodics, immunomodulatory agents, nicotine patches, products containing fish oils, or any investigation drugs that may interfere with the results of the study.

Mesalamine is administered at a daily dosage of 2.4 g/day or 4.8 g/day.

Patients are assessed at weeks 0, 3 and 6. The PGA score (physician's global assessment) is used for the assessment of both disease severity and efficacy. The components of the PGA scoring system include stool frequency, rectal bleeding, endoscopic findings, and the patient's functional assessment (PFA). Stool frequency, rectal bleeding, and endoscopic findings are rated on a scale from 0 to 3, indicating a range of normal to severe activity. In addition, the PFA was rated on a scale of 0 to 3, indicating a range of normal function to severe impairment. The patients may use electronic diaries to collect daily data for stool frequency, rectal bleeding, and the PFA. The investigators review each of these measurements at each visit. The PGA score is determined based on a combination of the clinical assessment scores and the investigators clinical judgment at weeks 0, 3 and 6. Overall improvement (“treatment success”) is defined as either complete remission or a clinical response to therapy. Complete remission is defined as normal stool frequency, no rectal bleeding, a well PFA score, normal endoscopy findings and a PGA score of 0. A clinical response to therapy is defined as improvement in the PGA score and at least one of stool frequency, rectal bleeding, PFA, and endoscopy results in conjunction with no worsening of other indicators.

A primary efficacy endpoint is overall improvement at week 6 in patients with moderately active ulcerative colitis (defined as a PGA score of 2). Secondary efficacy endpoints in patients with moderately active colitis include overall improvement at week 3, improvement from baseline in each of the clinical assessment score at weeks 3 and 6, overall improvement at week 6 in patients with ulcerative colitis limited to the left side of the colon, time to normalization of stool frequency, time to resolution of rectal bleeding, and change from baseline in the Ulcerative Colitis Disease Activity Index. A_{χ}² test may be used to standardize the data.

In one embodiment, at least about 59% moderately active ulcerative colitis or at least about 72% of patients with moderately active ulcerative colitis experience an overall improvement at week 6. In another embodiment, at least about 17% of patients with moderately active ulcerative colitis or at least about 20% of patients with moderately active ulcerative colitis experience complete remission at week 6.

In one embodiment, at least about 51% of patents with moderately active ulcerative colitis or at least about 61% of patients with moderately active ulcerative colitis experience an overall improvement at week 3.

In one embodiment, the median time to symptom resolution for stool frequency and rectal bleeding is about 9 days or about 16 days for patients with moderately active ulcerative colitis.

In one embodiment, at least about 40% of patients with mildly active ulcerative colitis or at least about 32% of patients with mildly active ulcerative colitis experience an overall improvement at week 6.

The invention is further illustrated by the following examples.

### EXAMPLE 1

### Dissolution Profiles for Mesalamine Tablets with Various Film Coatings

Core tablets comprising 400 mg of mesalamine were coated with various test films and subjected to dissolution profile measurements. In addition to the 400 mg mesalamine, the core tablets tested contained conventional excipients.

The test films were made of at least one copolymer of esters of acrylic and methacrylic acid, a glidant and optionally a plasticizer. Among the various copolymers used were a methacrylic acid-ethyl acrylate copolymer with a 1:1 ratio of free carboxyl groups:esters (for example, Eudragit® L30D-55) and an anionic copolymer based on methyl acrylate, methyl methacrylate and methacrylic acid, with a ration of about 1:10 of free carboxyl groups to esters (for example, Eudragit® FS 30 D).

The dissolution test used was in accordance with standard USP dissolution test procedures using a Type II apparatus (paddle), as follows. For each film coating, 6 tablets were used in the dissolution test.

Initially, disintegration of the coated tablets in 900 mL 0.1N HCl was tested for 2 hrs at 37° C.

The tablets were then removed from the 0.1N HCl medium for stage 2 dissolution testing in 900 mL pH 6.0 phosphate buffer in the paddle apparatus, with paddles stirring at 100 RPM at 37.5° C. for 1 hr. A 50 mL aliquot was removed to provide 5 mL samples for measuring the UV absorbance at 330 nm.

The pH of the dissolution buffer was raised to pH 7.2 with addition of 50 mL 2N NaOH. Paddle speed was reduced to 50 rpm. Dissolution at 37.5° C. was allowed to proceed for 1 hr before removal of a 10 mL aliquot for UV absorbance measurements at 332 nm.

The pH of the dissolution buffer was again raised, this time to pH 7.5 by adding 10 mL 2N NaOH. After 1 hr dissolution at 37.5° C. at a paddle speed of 50 rpm, a 10 mL aliquot was withdrawn for a UV absorbance measurement at 332 nm.

Weight percent dissolution of mesalamine at each pH is presented in Table 1 below. In Table 1, the column labeled “Polymer Ratio Time (% wt gain)” presents the polymers used in the film, the ratio of polymers in the film, time in hours that the film was dried, and the polymer weight gain of the tablet after coating in parenthesis. For example, “FS30D/L30D55 25:75 T24 (3%)” means that the film was made of FS30D and L30D55 at a weight ratio of 25 FS30D:75 L30D55, that the time of testing the dissolution profile of the tablet (Tt) was at t=24 hrs, and that the polymer weight gain on the tablet was 3%, calculated as a dried base. Additionally, in some rows, if drying was performed at room temperature, the designation “RT” follows the number of hours the film was dried. The % relative standard deviation (% RSD) of the replicate measurements at pH 6.0 is also presented in the final column.

TABLE 1
Dissolution Profile of mesalamine tablets with various film coatings

| Polymer Ratio Time (% wt gain) | 1.2 pH | 6.0 pH | 7.2 pH | 7.5 pH |
|---|---|---|---|---|
| FS30D/L30D55 25:75 T0 (3%) | 0 | 77 | 92 | 96 |
| FS30D/L30D55 25:75 T24 (3%) | 0 | 33 | 79 | 93 |
| FS30D/L30D55 25:75 T60 (3%) | 0 | 31 | 78 | 93 |
| FS30D/L30D55 25:75 T24 RT | 0 | 91 | 97 | 98 |
| (7%) | | | | |
| FS30D/L30D55 25:75 24 RT (2%) | 0 | 83 | 88 | 91 |
| FS30D/L30D55 25:75 24 RT (4%) | 0 | 24 | 68 | 87 |
| FS30D/L30D55 25:75 T16 (9%) | 0 | 43 | 88 | 95 |
| FS30D/L30D55 25:75 T24 (9%) | 0 | 34 | 90 | 98 |
| FS30D/L30D55 25:75 24 RT (2%) | 0 | 83 | 88 | 91 |
| FS30D/L30D55 25:75 24 RT (4%) | 0 | 24 | 68 | 87 |
| FS30D/L30D55 25:75 24 RT (6%) | 0 | 0 | 32 | 75 |
| FS30D/L30D55 15:85 T16 (9%) | 0 | 18 | 85 | 96 |
| FS30D/L30D55 15:85 T36 (9%) | 0 | 9 | 83 | 96 |
| FS30D/L30D55 15:85 T60 (9%) | 0 | 10 | 85 | 97 |
| FS30D/L30D55 15:85 T108 (9%) | 0 | 7 | 83 | 99 |
| FS30D/L30D55 15:85 48 RT (9%) | 0 | 46 | 88 | 94 |
| FS30D/L30D55 15:85 96 RT (9%) | 0 | 36 | 97 | 108 |
| FS30D/L30D55 15:85 72 RT | 0 | 19 | 84 | 95 |
| (11%) | | | | |
| FS30D/L30D55 15:85 96 RT | 0 | 4 | 82 | 94 |
| (11%) | | | | |
| FS30D/L30D55 15:85 48 RT | 0 | 5 | 84 | 95 |
| (13%) | | | | |
| FS30D/L30D55 15:85 T0 (8%) | 0 | 74 | 93 | 96 |
| FS30D/L30D55 15:85 T24 (8%) | 0 | 48 | 92 | 97 |
| FS30D/L30D55 15:85 T48 (8%) | 0 | 40 | 89 | 96 |
| FS30D/L30D55 15:85 T72 (8%) | 0 | 27 | 88 | 98 |
| FS30D/L30D55 15:85 T96 (8%) | 0 | 58 | 90 | 95 |
| FS30D/L30D55 15:85 T119 (8%) | 0 | 82 | 93 | 96 |
| FS30D/L30D55 20:80 T0 (7%) | 0 | 63 | 88 | 94 |
| FS30D/L30D55 20:80 T24 (7%) | 0 | 33 | 86 | 96 |
| FS30D/L30D55 20:80 T0 (8%) | 0 | 38 | 81 | 94 |
| FS30D/L30D55 20:80 T24 (8%) | 0 | 20 | 82 | 95 |
| FS30D/L30D55 20:80 T48 (8%) | | | | |
| FS30D/L30D55 15:85 T0 (8.5%) | 0 | 12 | 85 | 94 |
| FS30D/L30D55 15:85 T24 (8.5%) | 0 | 10 | 87 | 95 |
| FS30D/L30D55 15:85 T48 (8.5%) | 0 | 62 | 89 | 95 |
| FS30D/L30D55 15:85 T72 (8.5%) | 0 | 65 | 91 | 95 |
| FS30D/L30D55 15:85 T0 (8.5%) | 0 | 32 | 87 | 95 |
| FS30D/L30D55 15:85 T24 (8.5%) | 0 | 63 | 95 | 95 |
| FS30D/L30D55 15:85 T96 (8.5%) | 0 | 56 | 89 | 95 |
| FS30D/L30D55 15:85 T120 | 0 | 54 | 88 | 98 |
| (8.5%) | | | | |
| FS30D/L30D55 10:90 T0 (8%) | 0 | 63 | 92 | 96 |
| FS30D/L30D55 10:90 T24 (8%) | 0 | 80 | 94 | 96 |
| FS30D/L30D55 10:90 T48 (8%) | 0 | 11 | 85 | 95 |

### EXAMPLE 2

### Effect of a Plasticizer in the Film Coating on the Dissolution Profile.

The effect of the plasticizer triethyl citrate (TEC) content on the dissolution profile was also examined as a function of dried polymer ratio and drying conditions. Different weight percent concentrations of TEC were added to the film coating polymers at differing coating weight gain percentages. Dissolution profiles using TEC concentrations of 5, 7 and 10% are presented in Tables 2-4 below. The column labeled “Ratio of FS 30 D: L 30 D-55 Time (% wt gain)” presents the same information as described above for Table 1. Additionally, in some rows, the drying conditions, temperature and or humidity follows the number of hours the film was dried.

TABLE 2
Dissolution Profile of Mesalamine Tablets
with Film Coatings Including 5% TEC

| Ratio of FS 30 D:L 30 D-55 | | | | |
|---|---|---|---|---|
| Time (% wt gain) | pH 1.2 | pH 6.0 | pH 7.2 | pH 7.5 |
| 15:85 T24 (5%) | 0 | 87 | 98 | 100 |
| 15:85 T24 (7%) | 0 | 74 | 100 | 102 |
| 15:85 T24 (9%) | 0 | 61 | 96 | 101 |
| 15:85 T24 (11%) | 0 | 32 | 90 | 101 |
| 15:85 (9%) T48@RT | 0 | 46 | 88 | 94 |
| 15:85 (9%) T96@RT | 0 | 36 | 97 | 108 |
| 15:85 (11%) T60 RT | 0 | 19 | 84 | 95 |
| 15:85 (11%) T72@RT | 0 | 4 | 82 | 94 |
| 15:85 (13%) T48@RT | 0 | 5 | 84 | 95 |
| 25:75 (10%) T 24@RT | 0 | 0 | N/A | N/A |
| 15:85 (9%) T24@RT | 0 | 0 | N/A | N/A |
| 25:75 (2%) T24 RT | 0 | 83 | 88 | 91 |
| 25:75 (2%) T48 RT | 0 | 52 | 80 | 92 |
| 25:75 (4%) T24 RT | 0 | 24 | 68 | 87 |
| 25:75 (6%) T24 RT | 0 | 0 | 32 | 75 |
| 15:85 (9%) T16 40 C. | 0 | 18 | 85 | 96 |
| 15:85 (9%) T36 40 C. | 0 | 9 | 83 | 96 |
| 15:85 (9%) T60 40 C. | 0 | 10 | 85 | 97 |
| 15:85 (9%) T108 40 C. | 0 | 7 | 83 | 99 |
| 25:75 (3%) T0 RT | 0 | 77 | 92 | 96 |
| 25:75 (3%) T24 40 C. | 0 | 33 | 79 | 93 |
| 25:75 (3%) T120 40 C. | 0 | 31 | 78 | 93 |
| 15:85 (8%) T0 | 0 | 74 | 93 | 96 |
| 15:85 (8%) T24 40 C. | 0 | 48 | 92 | 97 |
| 15:85 (8%) T48 40 C. | 0 | 40 | 89 | 96 |
| 15:85 (8%) T72 40 C. | 0 | 27 | 88 | 96 |
| 15:85 (8%) T96 40 C. | 0 | 58 | 90 | 96 |
| 15:85 (8%) T119 40 C. | 0 | 82 | 93 | 96 |
| 30:70 (2%) T17 40 C. | 0 | 4 | 35 | 77 |
| 15:85 (8.5%) T0 | 0 | 12 | 85 | 94 |
| 15:85 (8.5%) T24 40 C. | 0 | 10 | 87 | 95 |
| 15:85 (8.5%) T48 40 C. | 0 | 62 | 89 | 95 |
| 15:85 (8.5%) T72 40 C. | 0 | 65 | 91 | 95 |
| 15:85 (8.5%) T day 60 C./75% RH | 0 | 55 | 92 | 98 |
| 15:85 (8.5%) T10 days 60 C./ | 0 | 15 | 84 | 98 |
| 75% RH | | | | |
| 10:90 (8%) T0 | 0 | 0 | N/A | N/A |
| 20:80 T0 (6%) | 0 | 86 | 96 | 98 |
| 20:80 T24 40 C. (6%) | 0 | 69 | 93 | 98 |
| 20:80 T72 40 C. (6%) | 0 | | | |
| 15:85 T0 (6%) | 0 | 87 | 97 | 97 |
| 15:85 T24 (6%) 40 C. | 0 | 79 | 95 | 97 |

TABLE 3
Dissolution Profile of Mesalamine Tablets with
Film Coatings Including 7% TEC

| Subcoat | Ratio | pH1.2 | pH6.0 | pH7.2 | pH7.5 |
|---|---|---|---|---|---|
| — | 20:80 T0 (7%) | 0 | 63 | 88 | 94 |
| — | 20:80 T24 (7%) | 0 | 33 | 86 | 96 |
| — | 20:80 T12days (7%) | 0 | 32 | 81 | 93 |
| — | 20:80 T0 (8%) | 0 | 38 | 81 | 94 |
| — | 20:80 T12days (8%) | 0 | 18 | 73 | 91 |
| — | 20:80 T0 (9%) | 0 | 1 | 65 | 89 |
| — | 25:75 T0 (3%) | N/A | 61 | N/A | N/A |
| — | 25:75 T24 (3%) | 0 | 5 | 33 | 71 |
| — | 25:75 T24 (4%) | 0 | 5 | 37 | 77 |
| — | 15:85 T0 (8.5%) | 0 | 32 | 87 | 95 |
| — | 15:85 T48 (8.5%) | 0 | 63 | 95 | 95 |
| — | 15:85 T96 (8.5%) | 0 | 56 | 89 | 95 |
| — | 15:85 T120 (8.5%) | 0 | 54 | 88 | 98 |
| PVP | 15:85 T0 (8.5%) | 0 | 17 | 86 | 94 |
| PVP | 15:85 T24 (8.5%) | 0 | 58 | 92 | 94 |
| PVP | 15:85 T0 (8%) | 0 | 70 | 92 | 95 |
| PVP | 15:85 T96 (8%) | 0 | 83 | 95 | 95 |
| — | 25:75 T0 (3%) | 0 | 94 | 92 | 95 |
| — | 25:75 T24 RT (30%) | 0 | 75 | 90 | 94 |
| — | 25:75 T24 40C (3%) | 0 | 52 | 69 | 83 |
| — | 20:80 TO (8%) | 0 | 2 | 75 | 93 |
| — | 20:80 T24 40C (8%) | N/A | N/A | N/A | N/A |
| — | 15:85 TO (8.5%) | 0 | 35 | 87 | 94 |
| — | 15:85 T24 40C (8.5%) | 0 | 3 | 84 | 97 |
| — | 15:85 T48 40C (8.5%) | N/A | N/A | N/A | N/A |

TABLE 4
Dissolution Profile of Mesalamine Tablets with
Film Coatings Including 10% TEC

| Subcoat | Ratio | pH1.2 | pH6.0 | pH7.2 | pH7.5 |
|---|---|---|---|---|---|
| — | 15:85 (10%) T16 40C | 0 | 31 | 85 | 94 |
| — | 15:85 (12%) T16RT | 0 | 51 | 88 | 96 |
| — | 15:85 (12%) T16 RT | 0 | 37 | 87 | 94 |
| — | 15:85 (14%) T16@RT | 0 | 12 | 83 | 92 |
| — | 15:85 (15%) T16 RT | 0 | 7 | 84 | 96 |
| — | 15:85 (17%) T16 RT | 0 | 2 | 83 | 94 |
| — | 25:75 (5%) T24 RT | 0 | 85 | 97 | 98 |
| — | 25:75 (5%) T36 RT | 0 | 58 | 92 | 96 |
| — | 25:75 (7%) T24 RT | 0 | 91 | 97 | 98 |
| — | 25:75 (9%) T24 RT | 0 | 34 | 90 | 98 |
| — | 25:75 (9%) T16 40C | 0 | 43 | 88 | 95 |
| — | 20:80 (6%) T0 | 0 | 77 | 94 | 98 |
| — | 20:80 (6%) T24 40C | 0 | 72 | 92 | 96 |
| — | 20:80 (8%) T0 | 0 | 9 | 75 | 95 |

More detailed investigation of dissolution at pH 6.0 was performed for a selection of film coated tablets. These results are presented in Tables 5 and 6 below.

TABLE 5
Dissolution at pH 6.0 of Mesalamine Tablets with
FS30D/L30D55 Film Coatings Including 7% TEC

| | Ratio FS30D/L30D55 | | | | |
|---|---|---|---|---|---|
| Subcoat | Time (% wt gain) | 15 min | 30 min | 45 min | 60 min |
| — | 15:85 T24 (7.0%) | 0 | 9 | 43 | 89 |
| — | 15:85 T24 (6.5%) | 0 | 26 | 65 | 99 |
| — | 15:85 T24 (7.0%) | 0 | 16 | 59 | 96 |
| — | 15:85 T24 (7.5%) | 0 | 26 | 59 | 90 |
| — | 15:85 T24 (8.0%) | 0 | 1 | 27 | 69 |
| — | 10:90 T24 (8.0%) | 0 | 0 | 3 | 19 |
| — | 10:90 T24 (10.0%) | 0 | 0 | 0 | 5 |
| — | 15:85 3 day (7%) | 4 | 59 | 100 | 104 |
| — | 15:85 5 day (7%) | 1 | 67 | 101 | 105 |
| — | 15:85 7 day (7%) | 1 | 45 | 94 | 102 |
| — | 15:85 10 day (7%) | 1 | 48 | 98 | 105 |
| — | 15:85 T3 day (8.0%) | 0 | 1 | 36 | 78 |
| — | 15:85 T5 day (8.0%) | 0 | 1 | 37 | 82 |
| — | 15:85 T7 day (8.0%) | 0 | 2 | 37 | 82 |
| — | 15:85 T10 day (8.0%) | 0 | 1 | 33 | 78 |

TABLE 6
Dissolution of Mesalamine Tablets with L30D55
Film Coatings including 7% TEC at pH 6.0

| Time Temp (% wt gain) | 15 min | 30 min | 45 min | 60 min |
|---|---|---|---|---|
| T2 day 40 C. (4%) | N/A | 18 | 68 | 84 |
| T2 day 40 C. (6%) | 0 | 12 | 63 | 91 |
| T2 day 40 C. (8%) | 0 | 1 | 30 | 75 |
| T3 day (10%) | 0 | 1 | 1 | 11 |
| T5 day (10%) | 0 | 0 | 0 | 0 |
| T7 day (10%) | 0 | 0 | 0 | 0 |
| T10 day (10%) | 0 | 0 | 0 | 2 |
| T3 day 40 C. (4%) | 21 | 81 | 95 | 98 |
| T3 day 40 C. (6%) | 0 | 15 | 67 | 91 |
| T3 day 40 C. (8%) | 0 | 0 | 15 | 59 |
| T5 day 60/75 C. (4%) | 1 | 51 | 77 | 86 |
| T5 day 60/75 C. (6%) | 0 | 2 | 38 | 66 |
| T5 day 60/75 C. (8%) | 0 | 0 | 5 | 37 |
| T3 day 40 C. 1 day 60/75 C. | 1 | 27 | 56 | 71 |
| (4%) | | | | |
| T3 day 40 C. 1 day 60/75 C. | 0 | 0 | 25 | 59 |
| (6%) | | | | |
| T3 day 40 C. 1 day 60/75 C. | 0 | 0 | 0 | 10 |
| (8%) | | | | |

### EXAMPLE 3

### Dissolution Profile of 800 mg of Asacol®

Two 400 mg tablets of Asacol® were subjected to the dissolution test described in Example 1. The measured wt % of mesalamine dissolved at each pH is presented in Table 7 below. At pH 6.0, no dissolved mesalamine was measured, while at pH 7.2 26% was measured as dissolved. At a pH of 7.5, virtually all (97%) was dissolved.

TABLE 7
Dissolution Profile of Asacol ®

| Sample | 1.2 pH | 6.0 pH | 7.2 pH | 7.5 pH |
|---|---|---|---|---|
| 800 mg Asacol ® (2 400 mg | 0 | 0 | 26 | 97 |
| Asacol ® tablets) | | | | |

### EXAMPLE 4

### Formulation of Mesalamine Core and Film Coating

An exemplary formulation for mesalamine tablets with a single polymer in the film coating was derived from the above experiments and is presented in the tables below.

### Formulation A:

Core Formulation

| No. | Material | mg/dose | % |
|---|---|---|---|
| 1 | Mesalamine | 400.0 | 78.43 |
| 2 | Lactose Monohydrate | 78.0 | 15.29 |
| 3 | Povidone K90 | 10.0 | 1.96 |
| 4 | Syloid 244FP | 3 | 0.59 |
| 5 | Explotab | 15 | 2.94 |
| 6 | Mag. Stearate | 4 | 0.78 |
| | Purified Water, USP* | 100* | — |
| 7 | Total | 510 | 100.0 |

*Water will be removed during drying stage

Eudragit Film Coating Formulation

| No. | Material | mg/dose | |
|---|---|---|---|
| 1 | Eudragit L30D55 | 12-20 | |
| 3 | TEC | 4-8 | |
| 4 | Talc | 8 | |
| 5 | Purified Water, USP* | 90* | — |
| 6 | Total | 30-44 | |

*Water will be removed during drying stage

Opadry II Beige Film Coating Formulation

| No. | Material | mg/dose |
|---|---|---|
| 1 | Opadry II Beige No. 85F97285 | 4-8 |
| 2 | Purified Water, USP* | 75* |
| | Total | 8-16 |

*Water will be removed during drying stage

### Formulation B:

A second exemplary formulation for mesalamine tablets, using two polymers in the film coating, was also derived from the above experiments. The formulation is presented in the tables below.

Core Formulation

| No. | Material | mg/dose | % |
|---|---|---|---|
| 1 | Mesalamine | 400.0 | 78.43 |
| 2 | Lactose Monohydrate | 78.0 | 15.29 |
| 3 | Povidone K90 | 10.0 | 1.96 |
| 4 | Syloid 244FP | 3 | 0.59 |
| 5 | Explotab | 15 | 2.94 |
| 6 | Mag. Stearate | 4 | 0.78 |
| | Purified Water, USP* | 100* | — |
| 7 | Total | 510 | 100.0 |

*Water will be removed during drying stage

Eudragit Film Coating Formulation

| No. | Material | mg/dose | |
|---|---|---|---|
| 1 | Eudragit L30D55 | 12-20 | |
| | Eudragit FS30D | 2-7 | |
| 3 | TEC | 4-8 | |
| 4 | Talc | 8 | |
| 5 | Purified Water, USP* | 90* | — |
| 6 | Total | 26-43 | |

*Water will be removed during drying stage

Opadry II Beige Film Coating Formulation

| No. | Material | mg/dose |
|---|---|---|
| 1 | Opadry II Beige No. 85F97285 | 4-8 |
| 2 | Purified Water, USP* | 75* |
| | Total | 8-16 |

*Water will be removed during drying stage

The terms “a” and “an” do not denote a limitation of quantity, but rather denote the presence of at least one of the referenced item. The term “or” means “and/or”. The terms “comprising”, “having”, “including”, and “containing” are to be construed as open-ended terms (i.e., meaning “including, but not limited to”) unless otherwise noted.

Recitation of ranges of values herein are merely intended to serve as a shorthand method of referring individually to each separate value falling within the range, unless otherwise indicated herein, and each separate value is incorporated into the specification as if it were individually recited herein. The endpoints of all ranges directed to the same component or property are inclusive and independently combinable.

All methods described herein can be performed in a suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention as used herein. Unless defined otherwise, technical and scientific terms used herein have the same meaning as is commonly understood by one of skill in the art to which this invention belongs. The terms wt %, weight percent, percent by weight, etc. are equivalent and interchangeable

Embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

We claim:

1. A mesalamine dosage form comprising
an effective amount of mesalamine,
wherein the dosage form has a dissolution profile such that greater than 5 wt % of the total weight of the mesalamine in the dosage form is released during a pH 6.0 portion of a dissolution test, the dissolution test comprising stirring in a pH 6.0 solution for 1 hour, followed by stirring in a pH 7.2 solution for an additional hour.

2. The mesalamine dosage form of claim 1, wherein the dosage form comprises a core comprising the effective amount of mesalamine, and a coating disposed on the core, the coating being soluble in aqueous solution at a pH of greater than or equal to 5.5 to less than 7.0.

3. The dosage form of claim 2, wherein the coating comprises
0-30% of a polymer soluble at pH>7.0; and
70-100% of a polymer soluble at pH≧5.5.

4. The dosage form of claim 2, wherein the coating comprises
0-30% of an anionic copolymer of methacrylic acid, methylacrylate and methyl methacrylate in which the ratio of free carboxyl groups to ester groups is about 1:10; and
70-100% of an anionic copolymer of methacrylic acid and ethyl acrylate in which the ratio of free carboxyl groups to ester groups is about 1:1.

5. The mesalamine dosage form of claim 1, wherein 5 wt % to about 35 wt % of the mesalamine dissolves in the pH 6.0 portion of the dissolution test, and about 65 wt % to 95 wt % of the mesalamine dissolves in the pH 7.2 portion of the dissolution test.

6. A mesalamine dosage form comprising
a core comprising an effective amount of mesalamine, wherein the core does not include a carbonate; and
a coating disposed on the core, the coating being soluble in aqueous solution at a pH of greater than or equal to 5.5 to less than 7.0, and
wherein the coating does not include a quaternary ammonium substituted acrylic polymer.

7. The mesalamine dosage form of claim 6, wherein the dosage form has a dissolution profile such that greater than 5 wt % of the total weight of the mesalamine in the dosage form is released during a pH 6.0 portion of a dissolution test, the dissolution test comprising stirring in a pH 6.0 solution for 1 hour, followed by stirring in a pH 7.2 solution for an additional hour.

8. The mesalamine dosage form of claim 7, wherein 5 wt % to about 35 wt % of the mesalamine dissolves in the pH 6.0 portion of the dissolution test, and about 65 wt % to 95 wt % of the mesalamine dissolves in the pH 7.2 portion of the dissolution test.

9. The dosage form of claim 6, wherein the coating comprises
0-30% of a polymer soluble at pH>7.0; and
70-100% of a polymer soluble at pH≧5.5.

10. The dosage form of claim 6, wherein the coating comprises
0-30% of an anionic copolymer of methacrylic acid, methylacrylate and methyl methacrylate in which the ratio of free carboxyl groups to ester groups is about 1:10; and
70-100% of an anionic copolymer of methacrylic acid and ethyl acrylate in which the ratio of free carboxyl groups to ester groups is about 1:1.

11. The dosage form of claim 6, wherein the coating comprises 3-15% by weight of the dosage form.

12. The dosage form of claim 7, wherein the dosage form has a dissolution profile such that at least 10 wt % of the total weight of the mesalamine in the dosage form is released during the pH 6.0 portion of the dissolution test.

13. The dosage form of claim 7, wherein the dosage form has a dissolution profile such that at least 20 wt % of the total weight of the mesalamine in the dosage form is released during the pH 6.0 portion of the dissolution test.

14. A method of treating an individual in need of treatment for ulcerative colitis, comprising administering to the individual the dosage form of claim 1.

15. The method of claim 14, wherein the dosage form comprises 400 mg to 800 mg of mesalamine.

16. The method of claim 14, wherein the dosage form is administered at a daily dosage of 2.4 g/day or 4.8 g/day.

17. The method of claim 14, wherein the dosage form comprises a core comprising the effective amount of mesalamine, and a coating disposed on the core, the coating being soluble in aqueous solution at a pH of greater than or equal to 5.5 to less than 7.0.

18. The method of claim 14, wherein the coating comprises
0-30% of a polymer soluble at pH>7.0; and
70-100% of a polymer soluble at pH≧5.5.
